(19) United States
(12) Patent Application Publication
(10) Pub. No.: US 2022/0265434 A1
(43) Pub. Date: Aug. 25, 2022

(54) IMPLANT SYSTEMS AND METHODS EMPLOYING A MOBILE GLENOSPHERE

(71) Applicant: IGNITE ORTHOPEDICS LLC, Winona Lake, IN (US)

(72) Inventors: Brian C. HODOREK, Winona Lake, IN (US); Matt J. PURDY, Winona Lake, IN (US); Russ M. PARROTT, Winona Lake, IN (US); J. Michael WIATER, Beverly Hills, MI (US); Anand M. MURTHI, Baltimore, MD (US); Matthew J. SMITH, Columbia, MO (US); Derek J. CUFF, Venice, FL (US); Andrew JAWA, Cambridge, MA (US); Luke AUSTIN, Haddonfield, NJ (US); Alec CUNNINGHAM, Fort Wayne, IN (US)

(73) Assignee: IGNITE ORTHOPEDICS LLC, Winona Lake, IN (US)

(21) Appl. No.: 17/662,903

(22) Filed: May 11, 2022

Related U.S. Application Data
(63) Continuation of application No. PCT/US2020/059020, filed on Nov. 5, 2020.
(60) Provisional application No. 62/935,432, filed on Nov. 14, 2019.

(51) Int. Cl. A61F 2/40

(52) U.S. Cl. CPC A61F 2/4081; A61F 2/4014; A61F 2002/4051; A61F 2002/30507

(57) ABSTRACT

A glenoid implant system for use in a reverse total shoulder replacement of a patient that includes a movable glenosphere, a baseplate, and a connector for movably connecting the glenosphere to the baseplate. The glenosphere and the baseplate are configured so that a central axis of the glenosphere is movable non-axially relative to a central axis of the baseplate. Further disclosed is a method having the steps of exposing and resecting a bone, securing a baseplate into the bone with the baseplate having a central axis, securing a glenosphere to the baseplate and non-axially moving the glenosphere relative to the central axis of the baseplate. Also disclosed is a method that includes the steps of rotating an arm in a medial direction, moving a glenosphere anteriorly relative to a baseplate attached to a glenoid, rotating the arm in a lateral direction, and moving the glenosphere posteriorly relative to the baseplate.

### CROSS-REFERENCE TO RELATED APPLICATION

This application is a continuation of PCT Application No. PCT/US2020/059020 filed on Nov. 5, 2020, entitled “Implant Systems and Methods Employing A Mobile Glenosphere” which claims priority benefit to U.S. Provisional Patent Application No. 62/935,432, filed Nov. 14, 2019, entitled “Implant Systems and Methods Employing a Mobile Glenosphere,” all of which are incorporated herein by reference in their entireties.

### FIELD OF THE INVENTION

The present disclosure relates generally to orthopedic surgery, implant systems, and methods for replacing an articulation joint, for example, in a repairing a shoulder joint. More specifically, but not exclusively, the present disclosure relates to implant systems and methods for reverse total shoulder replacement, which employ a mobile glenosphere movable non-axially relative to a baseplate attached to a glenoid.

### BACKGROUND TO THE INVENTION

In a typical anatomic total shoulder replacement, a damaged humeral head is replaced with a metal sphere, and a glenoid is replaced with a smooth plastic bearing surface. In a partial shoulder replacement, a damaged humeral head may only be replaced.

In a reverse total shoulder replacement, the ball and socket components of the shoulder joint are switched. In a conventional reverse total shoulder replacement, a glenosphere is fixed to the glenoid of the patient. The glenosphere mates with a humeral cup fixed to the humerus.

### SUMMARY OF THE INVENTION

Shortcomings of the prior art are overcome and additional advantages are provided through the provision, in one embodiment, of a glenoid implant system for use in a reverse total shoulder replacement of a patient. The glenoid implant system generally includes, for example, a glenosphere, a baseplate, and a connector. The glenosphere includes a body having a first portion and a second portion. The first portion has an articular surface, and the body defines a central axis extending from the first portion to the second portion. The baseplate is fixedly attachable to a glenoid of the patient. The baseplate has a body defining a central axis. The connector movably connects the glenosphere to the baseplate. The glenosphere and the baseplate are operably configured so that the central axis of the glenosphere is movable non-axially relative to the central axis of the baseplate.

In another embodiment, a glenoid implant system for use in a reverse total shoulder replacement of a patient includes, for example, a glenosphere having an articular surface, a baseplate fixedly attachable to a glenoid of the patient. The baseplate has a central axis, and a means for movably connecting the glenosphere to the baseplate so that the glenosphere is movable non-coaxially relative to the baseplate.

In another embodiment, a method includes, for example, surgically exposing and resecting a desired bone, securing a baseplate to the bone, the baseplate having a central axis, movably securing a glenosphere to the baseplate, and non-axially moving a central axis of the glenosphere relative to the central axis of the baseplate.

In another embodiment, a method includes, for example, moving an arm of a patient in a medial direction, moving a glenosphere anteriorly relative to a baseplate that is fixedly attached to a glenoid of the patient in response to moving the arm in the medial direction, moving the arm in a lateral direction, and moving the glenosphere posteriorly relative to the baseplate that is fixedly attached to the glenoid in response to moving the arm in the lateral direction.

### BRIEF DESCRIPTION OF THE DRAWINGS

The accompanying drawings, which are incorporated in and constitute a part of the specification, illustrate embodiments of the invention and together with the detailed description herein, serve to explain the principles of the disclosure. The drawings are only for purposes of illustrating preferred embodiments and are not to be construed as limiting the disclosure. It is emphasized that, in accordance with the standard practice in the industry, various features are not drawn to scale. In fact, the dimensions of the various features may be arbitrarily increased or reduced for clarity of discussion. The foregoing and other objects, features and advantages of the disclosure are apparent from the following detailed description taken in conjunction with the accompanying drawings in which:

FIG. 1 is a perspective view of an implanted reverse total shoulder implant system having a mobile glenosphere, according to an embodiment of the present disclosure;

FIG. 2 is an enlarged, exploded perspective view of the implanted reverse total shoulder implant system of FIG. 1, according to an embodiment of the present disclosure;

FIG. 3 is an enlarged, exploded perspective view of the glenoid implant system and humeral implant system of FIG. 1, according to an embodiment of the present disclosure;

FIG. 4 is an exploded perspective view of the glenoid implant system of FIG. 3, according to an embodiment of the present disclosure;

FIG. 5 is a front elevational view of the mobile glenosphere of FIG. 4, according to an embodiment of the present disclosure;

FIG. 6 is a right side elevational view of the mobile glenosphere of FIG. 5, according to an embodiment of the present disclosure;

FIG. 7 is a top view of the mobile glenosphere of FIG. 5, according to an embodiment of the present disclosure;

FIG. 8 is a bottom view of the mobile glenosphere of FIG. 5, according to an embodiment of the present disclosure;

FIG. 9 is a front elevational view of the baseplate of FIG. 4, according to an embodiment of the present disclosure;

FIG. 10 is a right side elevational view of the baseplate of FIG. 9, according to an embodiment of the present disclosure;

FIG. 11 is a top view of the baseplate of FIG. 9, according to an embodiment of the present disclosure;

FIG. 12 is a bottom view of the baseplate of FIG. 9, according to an embodiment of the present disclosure;

FIG. 13 is a perspective cross-sectional view taken along line 13-13 in FIG. 3 of the glenoid implant system, according to an embodiment of the present disclosure;

FIGS. 14 and 15 are perspective cross-sectional views of the glenoid implant system of FIG. 13 disposed in a first orientation and in a second orientation, respectively, according to an embodiment of the present disclosure;

FIG. 16 is an exploded, top perspective view of the humeral implant system of FIG. 3, according to an embodiment of the present disclosure;

FIG. 17 is an exploded, bottom perspective view of the humeral implant system of FIG. 3, according to an embodiment of the present disclosure;

FIGS. 18-20 are elevational side views of a shoulder of a patient with the humerus transitioning through external rotation;

FIGS. 21-23 are elevational side views of a shoulder of a patent with a prior art reverse total shoulder replacement having a fixed glenosphere with the humerus transitioning through external rotation;

FIGS. 24-26 are elevational side views of a shoulder of a patient with the reverse total shoulder implant system having the mobile glenosphere of FIG. 1 with the humerus transitioning through external rotation;

FIG. 27 is a perspective view of a glenoid implant system having a mobile glenosphere, according to an embodiment of the present disclosure;

FIG. 28 is an exploded, bottom perspective view of the glenoid implant system of FIG. 27, according to an embodiment of the present disclosure;

FIG. 29 is an exploded, top perspective view of the glenoid implant system of FIG. 27, according to an embodiment of the present disclosure;

FIG. 30 is a front elevational view of the mobile glenosphere of FIG. 27, according to an embodiment of the present disclosure;

FIG. 31 is a right side elevational view of the mobile glenosphere of FIG. 30, according to an embodiment of the present disclosure;

FIG. 32 is a top view of the mobile glenosphere of FIG. 30, according to an embodiment of the present disclosure;

FIG. 33 is a bottom view of the mobile glenosphere of FIG. 30, according to an embodiment of the present disclosure;

FIG. 34 is a cross-sectional view taken along line 34-34 in FIG. 33, according to an embodiment of the present disclosure;

FIG. 35 is a front elevational view of the baseplate of FIG. 27, according to an embodiment of the present disclosure;

FIG. 36 is a right side elevational view of the baseplate of FIG. 35, according to an embodiment of the present disclosure;

FIG. 37 is a top view of the baseplate of FIG. 35, according to an embodiment of the present disclosure;

FIG. 38 is a bottom view of the baseplate of FIG. 35, according to an embodiment of the present disclosure;

FIG. 39 is a flowchart of a surgical method, according to an embodiment of the present disclosure; and

FIG. 40 is a flowchart of a method of use of a mobile glenosphere, according to an embodiment of the present disclosure.

### DETAILED DESCRIPTION OF THE INVENTION

Generally stated, disclosed herein are implant systems and methods for orthopedic surgery for replacing an articulation joint, such as, for example, in repairing a shoulder joint of patient. The implant systems and methods may employ a mobile glenosphere, which is movable relative to a baseplate attached to the glenoid of the patient.

In this detailed description and the following claims, the words proximal, distal, anterior, posterior, medial, lateral, superior and inferior are defined by their standard usage for indicating a particular part of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

As used herein, the word “exemplary” or “illustrative” means “serving as an example, instance, or illustration.” Any implementation described herein as “exemplary” or “illustrative” is not necessarily to be construed as preferred or advantageous over other implementations. Moreover, in the present description, the terms “upper”, “lower”, “left”, “rear”, “right”, “front”, “vertical”, “horizontal”, and derivatives thereof shall relate to the disclosure as oriented in the first figure of each embodiment.

Similarly, positions or directions may be used herein with reference to anatomical structures or surfaces. For example, as the current implants, devices, systems and methods are described herein with reference to use with the bones of the shoulder, the bones of the shoulder and upper arm may be used to describe the surfaces, positions, directions or orientations of the implants, devices, systems and methods. Further, the implants, devices, systems and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, systems and methods, and the aspects, components, features and the like thereof, described and/or illustrated herein may be changed, varied, modified, reconfigured or otherwise altered for use or association with another side of the body for a same or similar purpose without departing from the spirit and scope of the disclosure. For example, the implants, devices, systems and methods, and the aspects, components, features and the like thereof, described herein with respect to the right shoulder may be mirrored so that they likewise function with the left shoulder and vice versa. Further, the implants, devices, systems and methods, and the aspects, components, features and the like thereof, disclosed herein are described with respect to the shoulder for brevity purposes, but it should be understood that the implants, devices, systems and methods may be used with other bones of the body having similar structures, for example the lower extremity, and more specifically, with the bones of the ankle, foot, and leg.

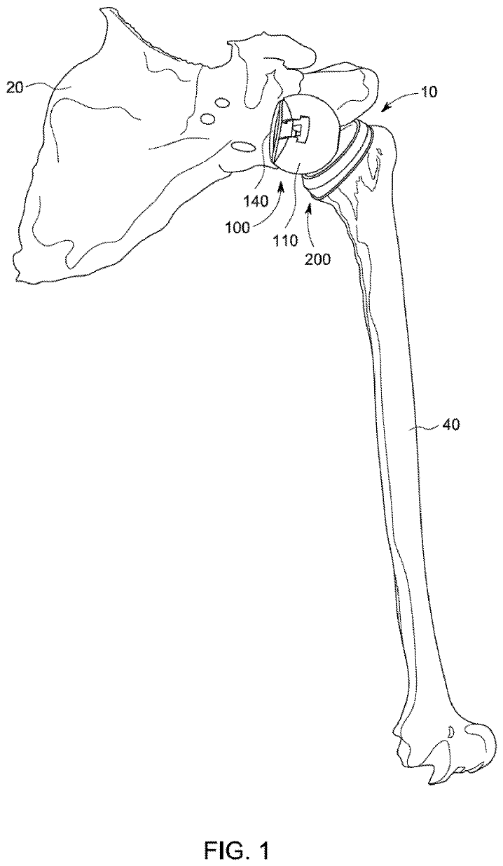

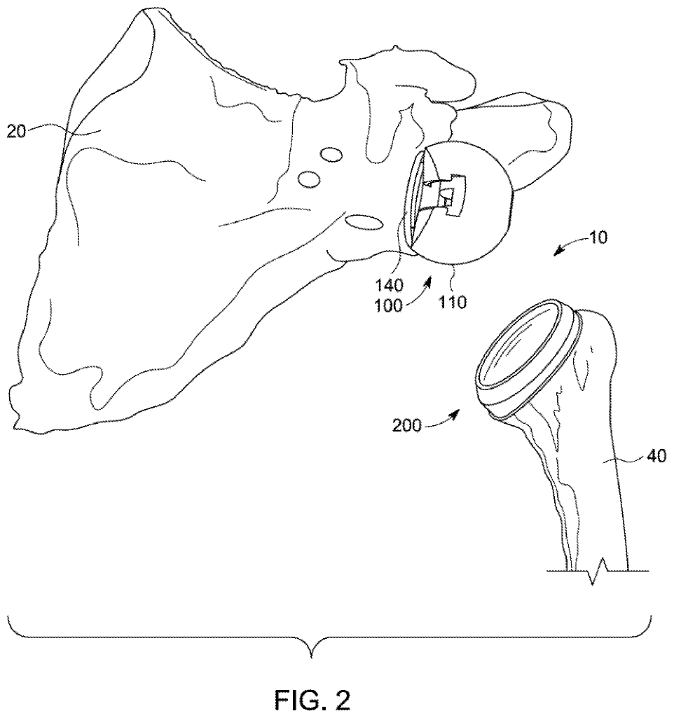

Referring to the drawings, wherein like reference numerals are used to indicate like or analogous components throughout the several views, and with particular reference to FIGS. 1 and 2, therein illustrated is an exemplary embodiment of a biocompatible prothesis implant system or reverse total shoulder implant system 10 for use in a reverse total shoulder replacement, according to an embodiment of the present disclosure. As will be appreciated from the description below, the technique of the present disclosure may allow a closer replication of the natural biomechanics of a normal shoulder compared to a conventional reverse total shoulder replacement.

In this exemplary embodiment, the reverse total shoulder implant system 10 may include a glenoid implant system 100 operably attached to a glenoid of a scapula 20 of a patient, and a humeral implant system 200 operably attached to a proximal portion of a humerus 40 of the patient. The glenoid implant system 100 may include a mobile glenosphere 110 and a baseplate 140. As described in greater detail below, the mobile glenosphere 110 may be operable to move in a manner that allows the humerus to function more similarly to its natural range of motion compared to conventional prosthesis implant systems for a reverse total shoulder replacement having a fixed glenosphere. For example, the reverse total shoulder implant system 10 may provide a closer replication of the natural biomechanics of a patient particularly in an internal and external rotation of the humerus, and in other joint motions of the arm and/or shoulder.

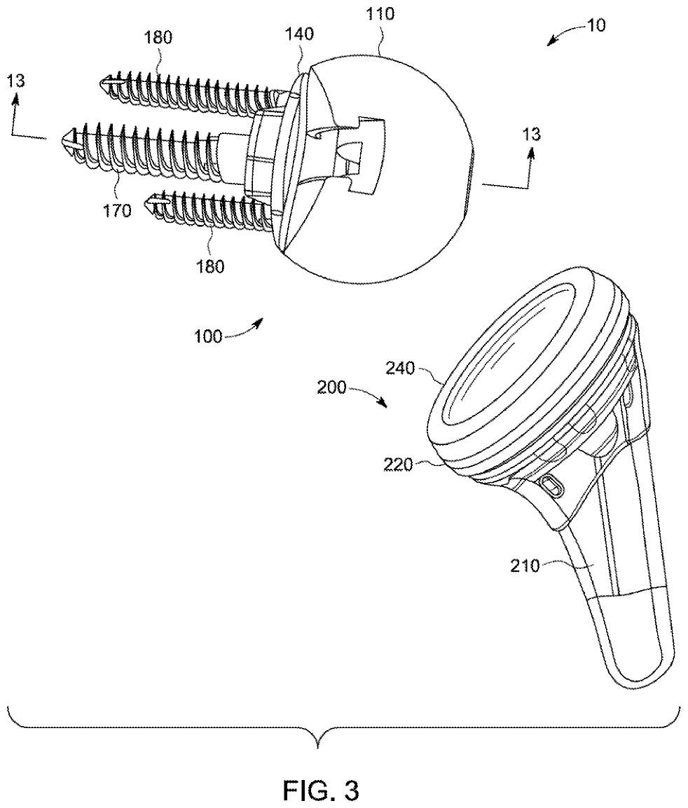

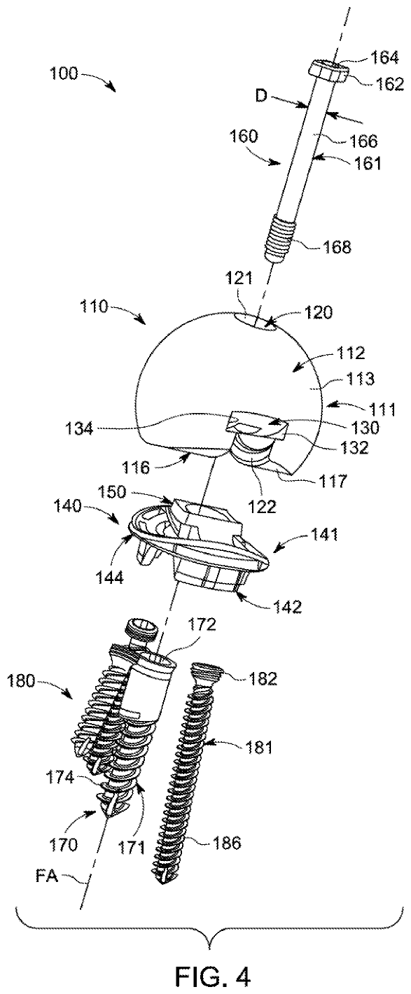

As shown in FIG. 3, the reverse total shoulder implant system 10 includes the glenoid implant system 100 and the humeral implant system 200. The glenoid implant system 100 may generally include the mobile glenosphere 110, the glenoid baseplate 140, a connector 160 (FIG. 4), a central screw 170, and at least one peripheral screw 180. The humeral implant system 200 may generally include a stem 210, coupling member or spacer 220, and a socket/bearing member 240.

With reference to FIGS. 4-8, the mobile glenosphere 110 may include a body 111 having a first portion 112 having a partial spherical surface 113 and a second portion 116 having spaced-apart curved surfaces 117. The first portion 112 is positioned opposite the second portion 116. The partial spherical surface 113 may have a radius R1 (FIG. 6). The partial curved surfaces 117 may have a radius R2 (FIG. 6). Radius RI may be less than radius R2. The radius of the partial spherical surface may be about 18 mm (millimeters), or between about 15 mm and about 26 mm. The radius of the spaced-apart surfaces may be about 26 mm, or between about 18 mm and about 40 mm.

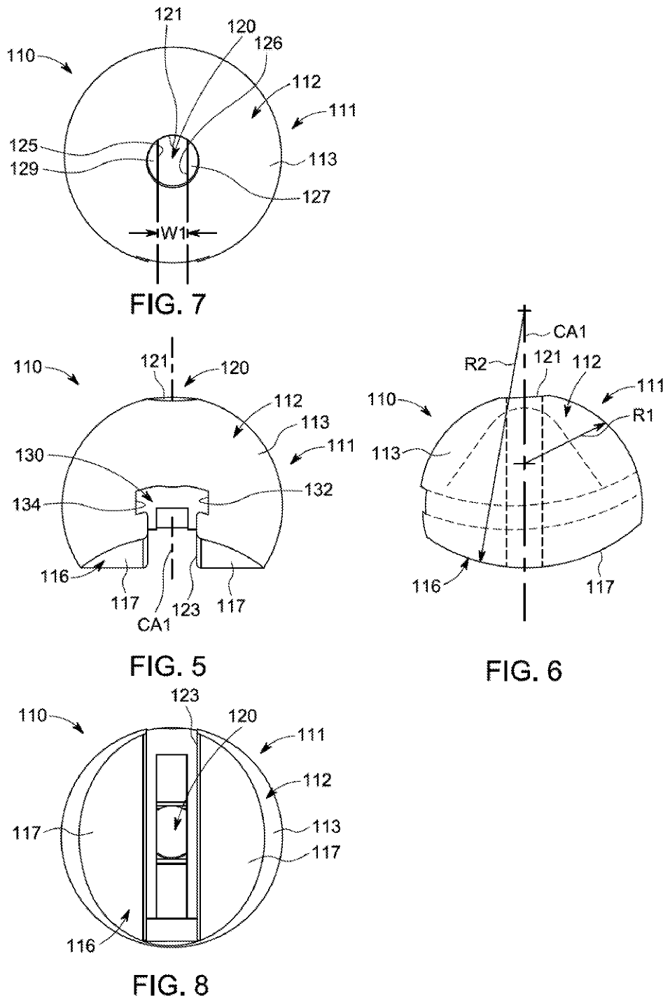

The mobile glenosphere 110 may include a central passageway 120 defining a central axis CA1 extending through the body 111 from the first portion 112 to the second portion 116. For example, the passageway 120 may include a circular opening 121 opening onto the partial spherical surface 113, and an elongate opening 123 (FIGS. 5 and 8) opening onto the curved surfaces 117 of the second portion 116. Body 111 of the mobile glenosphere 110 further defines a curved T-shaped track 130 disposed across the first portion 112 of the body 111. As described below, the configuration of the passageway 120 and the T-shaped track 130 allows the mobile glenosphere 110 to be movable relative to the baseplate 140.

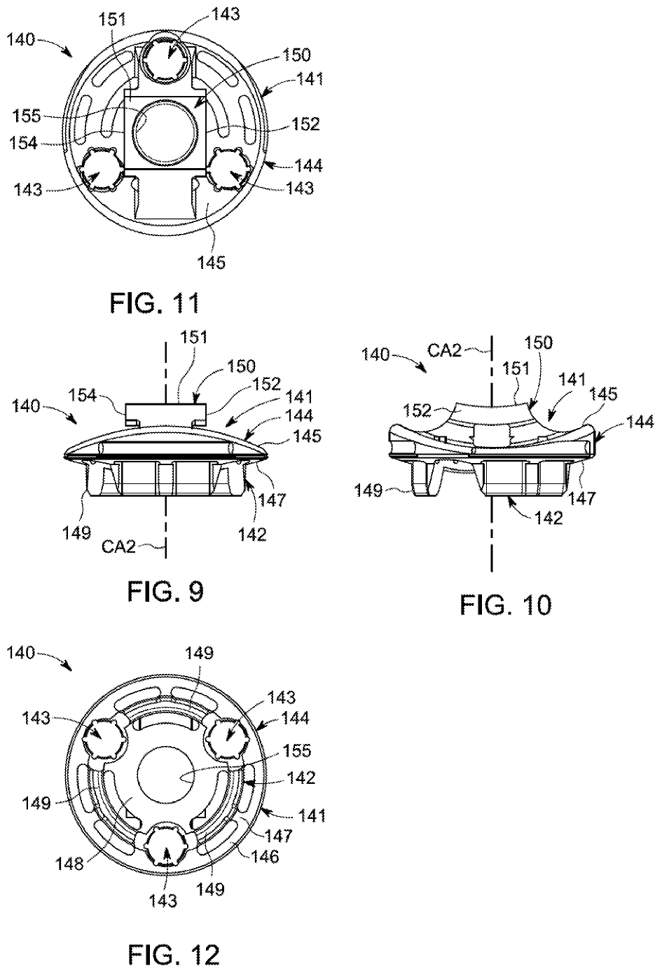

With reference to FIGS. 4 and 9-12, the baseplate 140 includes a body 141 having a guide portion 150, an implant portion 142, and a middle portion 144 disposed therebetween. The middle portion 144 of the baseplate 140, as shown in FIGS. 9-12, may have a general disc-shaped configuration having a first surface 145 and an opposite second surface 147. The middle portion 144 may include, for example, a constant or continuous exterior ring 146 (FIG. 12) surrounding a constant or continuous interior ring 148 (FIG. 12). The second portion 142 may include a plurality of discrete downwardly-depending arcuate keels, ridges, or protrusions 149.

The guide portion 150 of the baseplate 140 may define a first outwardly-extending curved flange 152 and a second outwardly-extending curved flange 154. As described below, the guide portion 150 and curved T-shaped track 130 (FIG. 4) of the mobile glenosphere 110 may be operably sized and configured so that the mobile glenosphere 110 may be slidably restrained on the guide portion 150 of the baseplate 140.

A central bore 155 may extend through the baseplate 140 from an outer surface 151 of the guide portion 150 to the second surface 147 of the middle portion 144. A plurality of through holes 143 may extend through the middle section 144 from the first surface 145 to the second surface 147. The through-holes 143 may, for example, be positioned between the plurality of downwardly-depending arcuate keels, ridges, or protrusions 149. The through-holes may be threaded through-holes.

With reference again to FIG. 4, the connector 160 of the mobile glenosphere 110 to the baseplate 140 may include an elongate body 161 having a proximal portion 162, a central portion 166, and a distal portion 168. The central portion 166 may have a cylindrical configuration having an outer diameter D. The proximal portion 162 may define an enlarged proximal cylindrical head 162 having a proximal recess 164. The proximal recess 164 may be, for example, a drive feature for engaging a tool for inserting or removing the connector 160. The distal portion 168 may include external threads configured or sized and shaped to operably connect to the central screw 170.

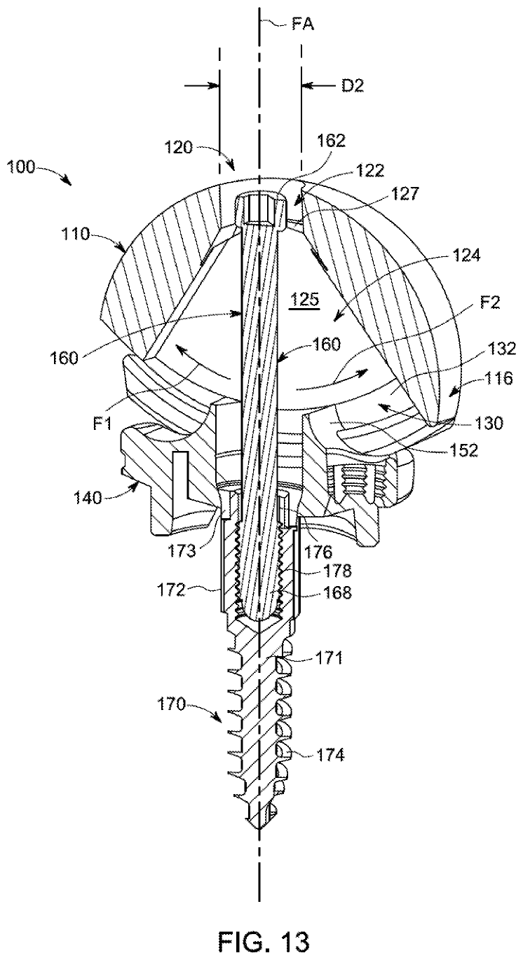

The central screw 170, as shown in FIGS. 4 and 13, may include an elongate body 171 having a proximal portion 172 and a threaded portion 174. As shown in FIG. 13, the proximal portion 172 may include an enlarged tapered flange 173 having, for example, a diameter larger than the diameter of the main portion of the proximal portion 172 of the central screw 170. The proximal portion 172 may also include a first section 176 and a second section 178 extending into the proximal portion 172. The first section 176 may have, for example, a drive feature for engaging a tool for inserting or removing the central screw 170. The second section 178 may be, for example, internally threaded for receiving the corresponding threaded portion 168 of connector 160. The proximal portion 172 may have, for example, a texture or coating to provide for porous fixation. The proximal portion 172 may be, for example, configured or sized and shaped to conserve bone. The distal threaded section 174 may be, for example, threaded to engage a patient's bone to secure the baseplate 140 to the patient's bone, such as, the glenoid. The combination of the central screw 170 and connector 160 defines a fixation axis FA, also shown in FIG. 4.

With reference still to FIG. 4, the at least one peripheral screw 180 may include an elongate body 181 having a head portion 182 and a screw thread portion 186. The head portion may include a drive opening recessed into the head portion 182 and outer threads disposed around the head portion. The at least one peripheral screw 180 may be, for example, three peripheral screws 180 as shown in the depicted embodiment, although alternative numbers of peripheral screws 180 are also contemplated to correspond to the number of peripheral bores 143 (FIG. 12) in the baseplate 140. The peripheral screws 180 may be inserted through the peripheral bores 143 (FIG. 12) in the baseplate 140 (FIG. 12) to engage a patient's bone, such as, the glenoid, to assist with securing the baseplate 140 to the patient's bone.

With reference again to FIG. 13, the passageway 120 of the mobile glenosphere 110 may include a cylindrical passageway portion 122, an expanded passageway portion 124 defined by parallel spaced apart sidewall 125 and sidewall 126 (FIG. 7) that angularly enlarge from cylindrical passageway portion 122 as it approaches and opens onto the T-shaped track 130. The cylindrical passageway portion 122 may have a diameter D2 sized to receive the proximal portion 162 of connector 160. The angularly expanded passageway portion 124 may have a constant width W1 (FIG. 7) disposed between sidewall 125 and sidewall 126 (FIG. 7). Width W1 (FIG. 7) is sized less than diameter D2 to define spaced apart curved stops 127 and 129 (FIG. 7) disposed between cylindrical passageway portion 122 and the expanded passageway portion 124 of passageway 120. The width W1 is sized to receive connector 160 therebetween with the proximal portion 162 of connector 160 sized to restrain the mobile glenosphere 110 axially relative to baseplate 140 along axis FA. The second portion 116 of the mobile glenosphere 110 may include opposite curved recesses 132 and 134 (FIGS. 4 and 5), which is supportable and guideable on the outwardly extending sides 152 and 154 (FIG. 4) of T-shaped track 130 of baseplate 140.

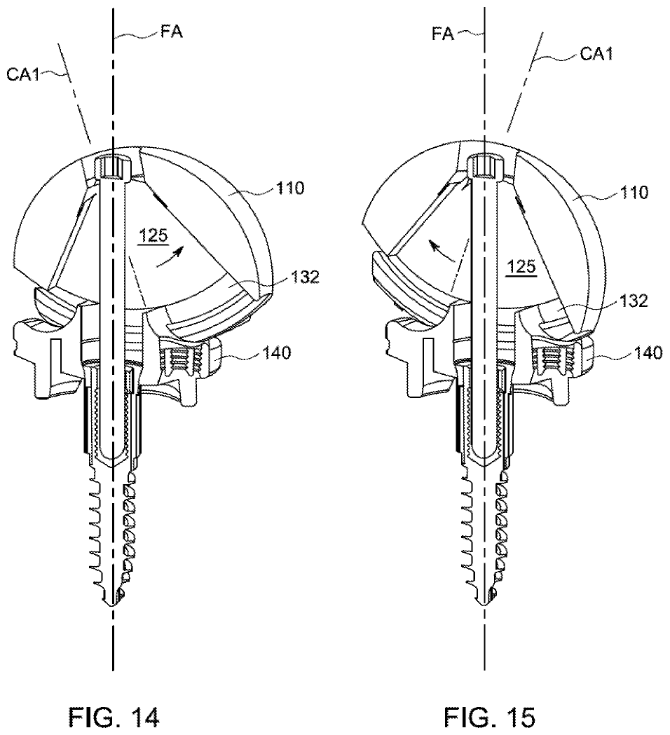

As shown in FIG. 13, the glenoid implant system 100 is operably configured and connected in this illustrated embodiment having means for movably connecting the mobile glenosphere 110 to the baseplate 140 so that the mobile glenosphere 110 is movable non-axially relative to the central axis of the baseplate 140 and the glenoid. It will be appreciated that the mobile glenosphere 110 is provided with clearance relative to the connector 160 so that the mobile glenosphere 110 movable relative to the connector 160. For example, the mobile glenosphere 110 may be angularly rotatable in a first direction F1 relative to the fixation axis FA and in the baseplate 140, and in a second direction F2 relative to the fixation axis FA and the baseplate 140. For example, the mobile glenosphere is movable so that the central axis CA1 of the mobile glenosphere 110 is movable from an axially aligned position with the fixation axis FA (shown in FIG. 13) to non-axial positions shown in FIGS. 14 and 15. As observed in FIGS. 14 and 15, the central axis CA1 of the mobile glenosphere 110 is tiltable relative to the fixation axis FA. In this illustrated embodiment, due to the parallel sidewall 125 and sidewall 127 (FIG. 4) and curved recesses 132 and 134 (FIG. 4), the central axis CA1 of the mobile glenosphere 110 is tiltable along a plane from the first position to the second non-coaxial position. In this illustrated embodiment, the mobile glenosphere 110 is tiltable over an angle of about 90 degrees. In other embodiments, the mobile glenosphere 110 may be movable over an angle of 10 degrees, 20 degrees, 30 degrees, 45 degrees, 60 degrees, 70 degrees, or 90 degrees, a range between 10 degrees and 30 degrees, a range between 20 degrees and 50 degrees, or a range between 30 degrees and 60 degrees.

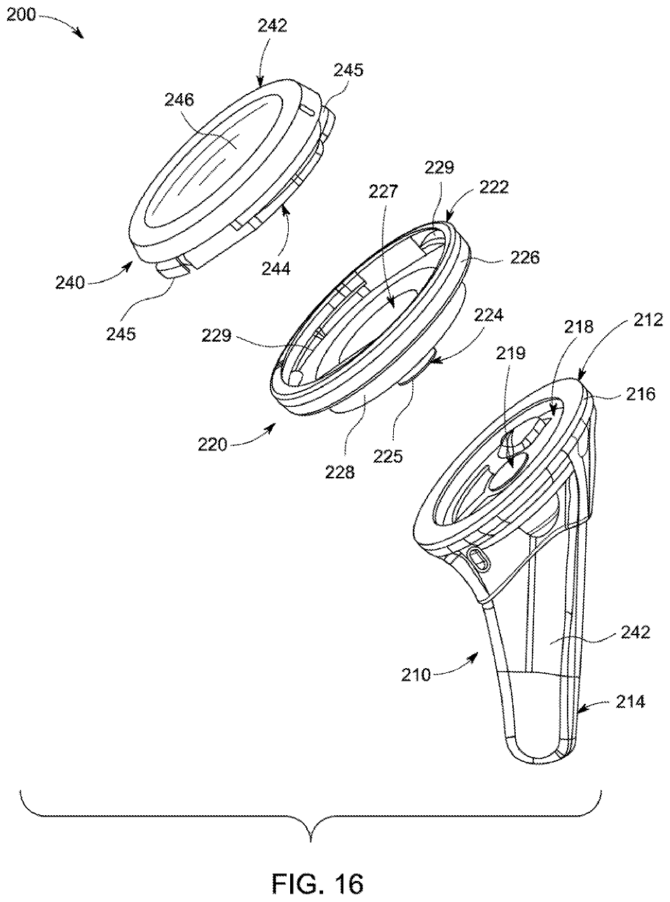

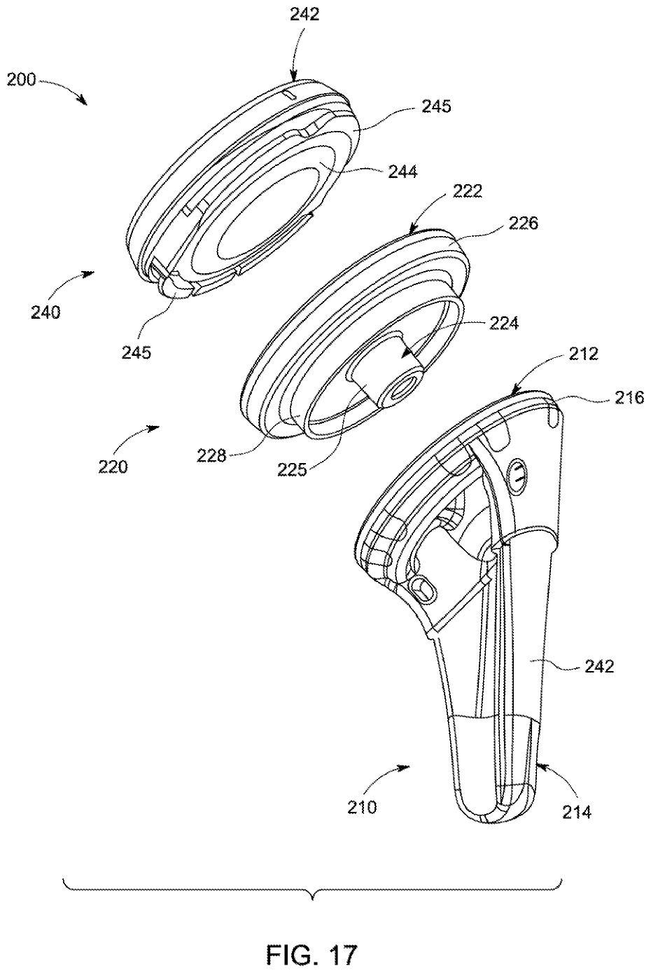

FIGS. 16 and 17 illustrate the humeral implant system 200, according to an embodiment of the present disclosure. In this illustrated embodiment, the humeral implant system 200 may include a stem component 210, a coupling member or spacer 220, and a socket/bearing member 240.

The stem component 210 may include a first end 212 and a second end 214. The stem component 210 may include a plate or base 216 and a stem 242. The base 216 may have a large ring or surface area to assist with fixation, for example, the base 216 may contact cancellous bone to provide better support for the humeral implant system 200. The base 216 may include a recess 218 and an opening 219 extending into the base 216 from the first end 212.

The coupling member 220 may include a first end 222 and a second end 224. The coupling member 220 includes a base member 226, an extension member 228, and a protrusion or extension member 225. The base member 226 includes a recessed region 227 inset into the coupling member 220 from the first end 222 and forming an interior side wall surrounding the recessed region 227. The interior side wall may include, for example, a circumferential groove portion 229. The protrusion or extension member 225 is receivable in the opening 219 in the stem 210.

The socket/bearing member 240 may include a first end 242 and a second end 244. The first end 242 includes an articulating surface 246 recessed into the socket member 240. The socket member 240 also includes engagement flanges 245 extending outwardly. The engagement flanges 245 may be, for example, operable to mattingly engage and be received in the recessed grooves of the coupling member 220. For example, the engagement flanges 245 of the socket member 240 may align with the circumferential grooves 229 of the coupling member 220 when the socket member 240 is inserted into the coupling member 220 and locked in place upon rotation of the socket member 240 relative to the coupling member 220.

As described above, the technique of the present disclosure may allow more closely replicating the natural biomechanics of a normal shoulder compared to a conventional reverse total shoulder replacements. In particular, the technique of the present disclosure may provide a reverse total shoulder replacement, wherein the proximal portion of the humerus more closely corresponds to the movement of a normal shoulder, especially during internal rotation (an arm being twisted inwardly), external rotation (the arm being twisted outwardly), and other joint motions of the arm and/or shoulder.

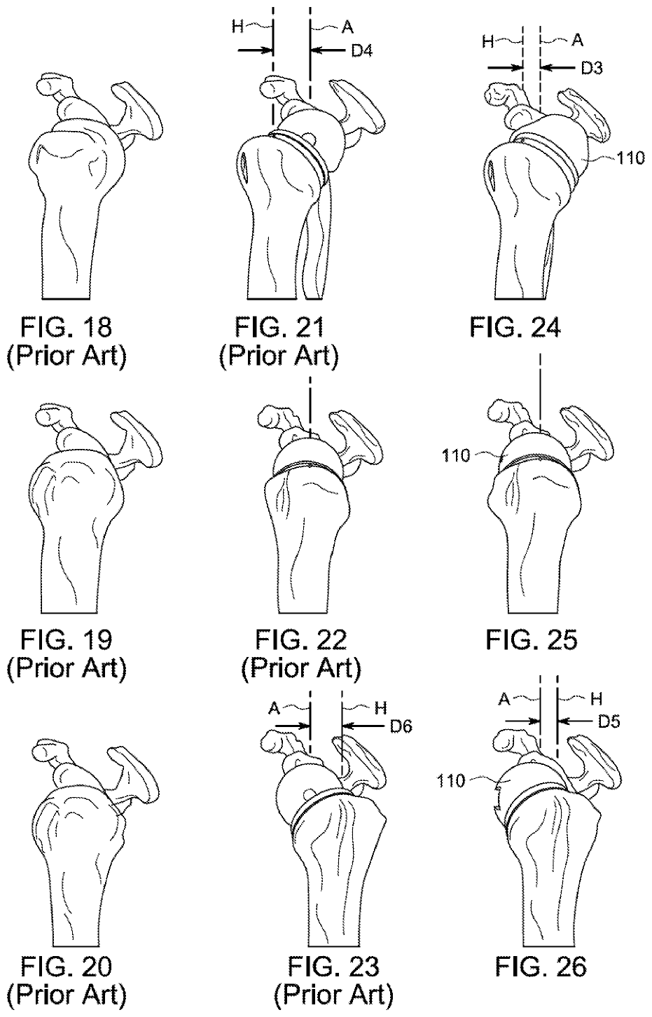

FIGS. 18-20 illustrate a normal shoulder of a patient transitioning through external rotation of the humerus, FIGS. 21-23 illustrate the shoulder of the patient having a conventional reverse total shoulder replacement transitioning through external rotation of the humerus, and FIGS. 24-26 illustrate the shoulder of the patient having a reverse total shoulder replacement that includes the reverse total shoulder implant system 10 with the mobile glenosphere 110 of the present disclosure transitioning through external rotation of the humerus. With reference to FIGS. 18-20, when a native humeral head internally and externally rotates within the glenoid cavity, it rotates about its articular centerpoint but also slides within the glenoid cup as it rotates. As a result, the centerpoint of the articular surface stays in roughly the same zone in three dimensional space through internal/external rotation of the humerus. With reference to FIGS. 21-23, during internal/external rotation, the humeral cup translates around the glenosphere since these components are fixed to the bones they are implanted in. As a result, the humeral articular centerpoint has excessive anterior/posterior and superior/inferior movement which is not conducive to the soft tissue and natural motion and can result in limited range of motion, soft tissue and bony impingement, and stretching of the soft tissue.

As is observed in FIGS. 24-26, the mobile glenosphere 110 moves posteriorly when the patient's arm is laterally rotated, and the mobile glenosphere 110 moves anteriorly when the patient's arm is medially rotated. In addition, the technique of the present disclosure employing the mobile glenosphere 110 as illustrated FIGS. 24-26 results in an anterior to a posterior movement of the humerus during a transition from medial rotation to lateral rotation that is reduced compared to an anterior to posterior movement of the humerus during a transition from medial rotation to lateral rotation employing a conventional technique with a fixed glenosphere as illustrated in FIGS. 21-23.

For example, in the technique of the present disclosure as shown in FIG. 24, medial rotation results in an axis H of a humerus being disposed anteriorly a distance D3 from an axis A aligned with a central axis of the baseplate or through the center of the glenoid. With a conventional reverse total shoulder replacement as shown in FIG. 21, medial rotation results in an axis H of a humerus being disposed anteriorly a distance D4 from an axis A aligned with a central axis of the baseplate or through the center of the glenoid. As illustrated, the distance D3 (FIG. 24) is less than the distance D4 (FIG. 21).

In addition, in the technique of the present disclosure as shown in FIG. 26, lateral rotation results in the axis H of a humerus being disposed posteriorly a distance D5 from the axis A aligned with a central axis of the baseplate or through the center of the glenoid. With a conventional reverse total shoulder replacement as shown in FIG. 23, lateral rotation results in the axis H of a humerus being disposed posteriorly a distance D6 from the axis A aligned with the central axis of the baseplate or through the center of the glenoid. As illustrated, the distance D5 (FIG. 26) is less than the distance D6 (FIG. 23).

From the present disclosure, the mobile glenosphere 110 can translate, pivot, roll, or otherwise move relative to the baseplate 140 below it, which is attached to the glenoid. This allows the shoulder joint to mimic the natural anatomic motion yet provide the benefits of a reverse shoulder arthroplasty. The mobile glenosphere 110 may be movable in relation to the rigidly fixed baseplate 140. While a mobile glenosphere may include a curved T-shaped track, it will be appreciated that other configurations of the track and baseplate guide may be employed. For example, the track may be a straight track having straight grooves and the baseplate guide may have a straight guide having straight sides. In other configurations, a track and guide may have a dovetail configuration or other suitable configurations. In still other embodiments, the track and guide may be reversed.

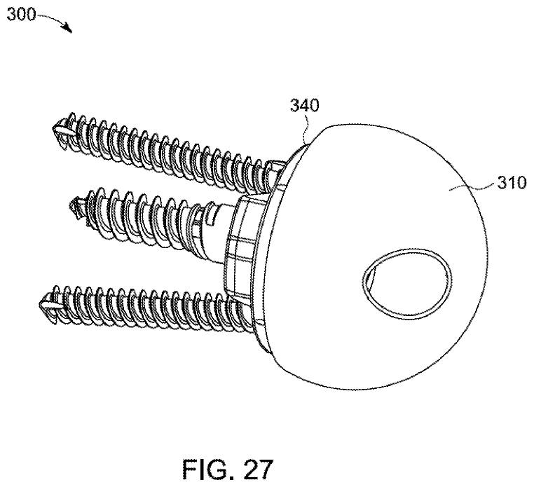

With reference to FIG. 27, therein illustrated is an exemplary embodiment of a glenoid implant system 300 operable in a biocompatible prothesis implant system or reverse total shoulder implant system for use in a reverse total shoulder replacement, according to an embodiment of the present disclosure. For example, a biocompatible reverse total shoulder implant system may include the glenoid implant system 300 operably attached to a glenoid of a scapula of a patient and a humeral implant system such as the humeral implant system 200 operably attached to a proximal portion of a humerus of the patient. As will be appreciated from the description below, the technique of the present disclosure may result in the replication of the natural biomechanics of a normal shoulder compare to a conventional reverse total shoulder replacement.

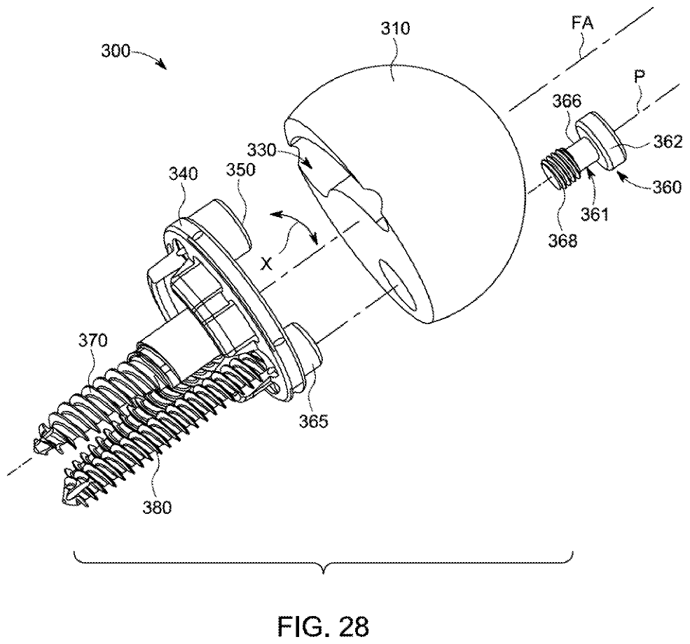

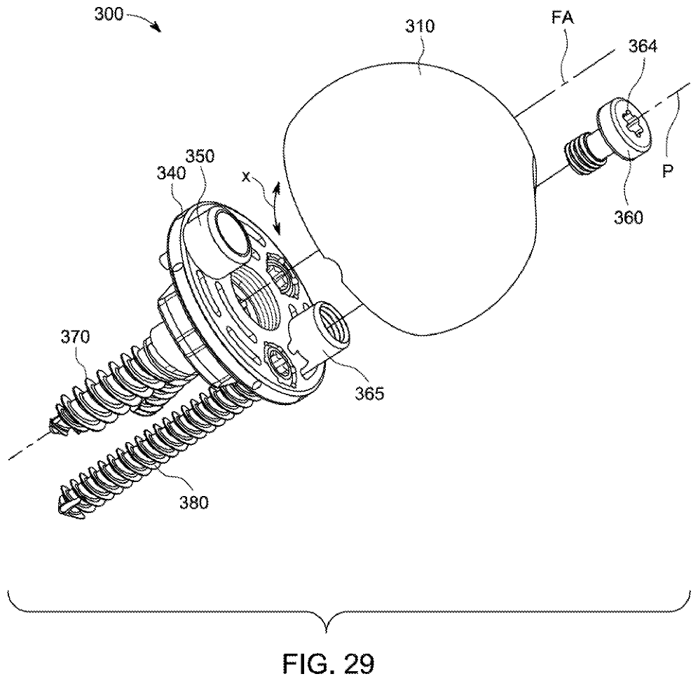

In this exemplary embodiment, as shown in FIGS. 28 and 29, the glenoid implant system 300 is operably configured and connected in this illustrated embodiment having means for movably connecting a mobile glenosphere 310 to a baseplate 340 so that the mobile glenosphere 310 is movable non-axially relative to the central axis of the baseplate 340 and the glenoid. For example, the mobile glenosphere 310 may include the mobile glenosphere 310, the baseplate 340 having guide 350 and a post 365, a connector 360, a central screw 370, and at least one peripheral screw 380. As described in greater detail below, the mobile glenosphere 310 may be operable to move in a manner, e.g., pivot about a central axis CA3 (FIGS. 31-34) of the glenosphere 310 about a pivot axis P in the direction of curved double headed arrow X, shown in FIGS. 28 and 29, that allows the humerus to mimic the natural range of motion compared to conventional prosthesis implant systems for a reverse total shoulder replacement having a fixed glenosphere. For example, a reverse total shoulder implant system with the mobile glenosphere 310 may more closely replicate the natural biomechanics of a patient particularly in lateral rotation to medial rotation, from medial rotation to lateral rotation, and other joint motions of the shoulder and/or arm.

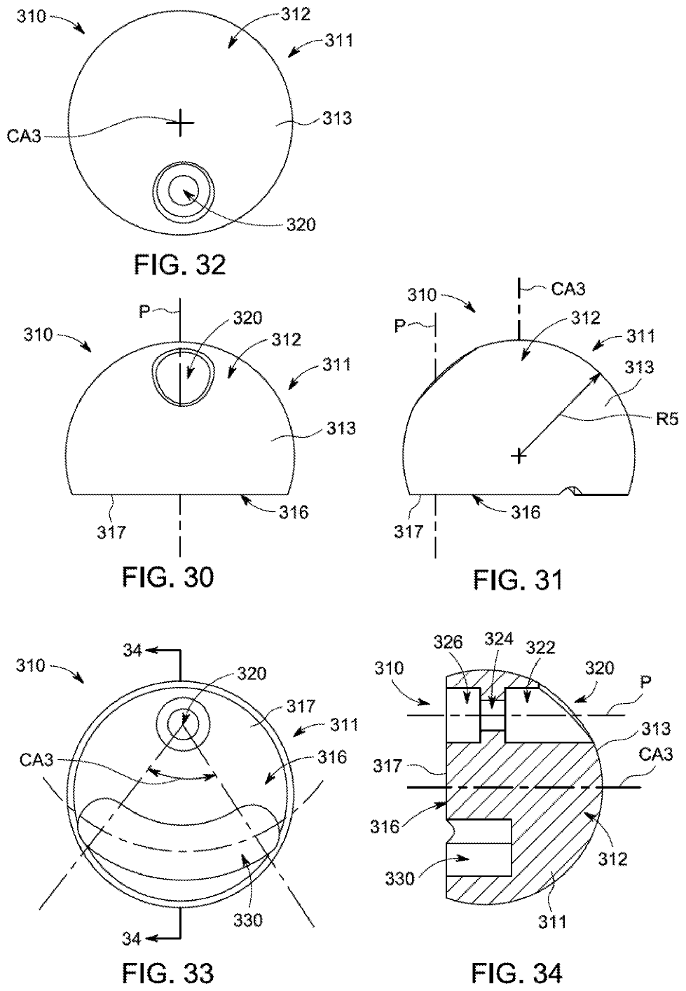

With reference to FIGS. 30-34, the glenosphere 310 may include a body 311 having a first portion 312 having a partial spherical surface 313 and a second portion 316 having a flat surface 317. The first portion 312 is positioned opposite the second portion 316. The partial spherical surface 313 may have a radius R5 (FIG. 31) that may correspond to the radius of the concave surface of a humeral implant system 200.

The glenosphere 310 may include an offset passageway 320 defining the offset axis P (FIGS. 30 and 31) extending through the body 311 from the first portion 312 to the second portion 316. For example, passageway 320 may include a first portion 322 opening onto first surface 313, a second portion 324, and a third portion 326 opening onto second surface 317. Body 311 of glenosphere 310 may further include a curved recessed slot 330 (FIGS. 33 and 28) extending into second portion 312 of body 311. As described below, the passageway 320, the connector 360, the post 365, and the curved recessed slot 330 maybe sized to provide proper clearance to allow the glenosphere 310 to be pivotably and non-axially movable relative to the baseplate 340 (FIG. 29).

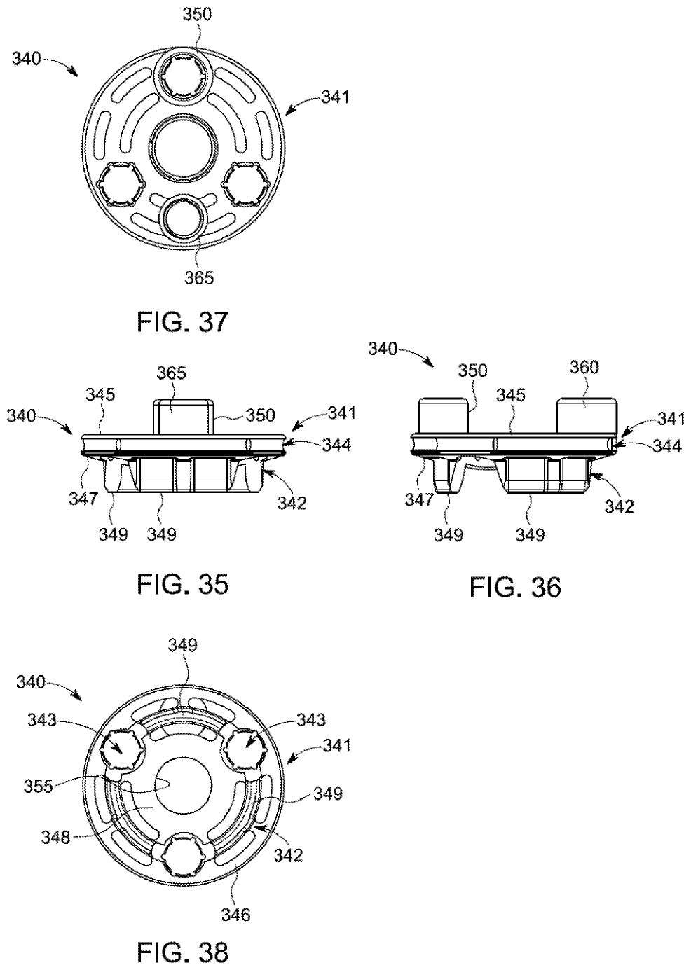

With reference to FIGS. 35-38, the baseplate 340 may include a body 341 having a first portion or an implant portion 342 and a second portion 344. The guide 350 may be a hollow cylindrical guide and the post 365 may be a hollow cylindrical post. The second portion 344 of the baseplate 340, as shown in FIGS. 35-38, may have a general disc-shaped configuration having a first surface 345 such as a flat surface and an opposite second surface 347. The second portion 344 may include, for example, a constant or continuous exterior ring 346 (FIG. 38) surrounding a constant or continuous exterior ring 348 (FIG. 38). The second portion 344 may include a plurality of discrete downwardly-depending arcuate keels, ridges, or protrusions 349.

With reference again to FIG. 28, the guide 350 of the baseplate 340 may define an outer cylindrical surface. As described below, the guide 350 and curved recess groove 330 of the glenosphere 310 may be operably sized and configure so that the glenosphere 310 may be pivotally and rotatably restrained relative to the baseplate 340.

With reference to FIGS. 35-38, a central bore 355 (FIG. 38) may extend through the baseplate 340 from an outer surface 345 of first portion 344 to the second surface 347 of the second portion 344 for receiving the central screw 370 (FIGS. 28 and 29) in a similar manner as described above with reference to baseplate 140 (FIG. 13).

A plurality of through holes 343 (FIG. 38) may extend through the second section 344 from the first surface 345 to the second surface 347. The through holes 343 (FIG. 38) may, for example, be positioned between the plurality of downwardly-depending arcuate keels, ridges, or protrusions 349. The through holes 343 (FIG. 38) may be threaded through holes for receiving the peripheral screws in a similar manner as described above with reference to baseplate 140 (FIG. 13).

With reference again to FIG. 28, the connector 360 may include a body 361 having a proximal portion 362, a central portion 366, and a distal portion 368. The central portion 366 may have a cylindrical configuration. The proximal portion 362 may define an enlarged proximal cylindrical head 362 having proximal recess 364 (FIG. 29). The proximal recess 364 (FIG. 29) may be, for example, a drive feature for engaging a tool for inserting or removing the connecting post or connector 360. The distal portion 368 may include external threads configured or sized and shaped to operably connect to internal threads in the post 365.

The central screw 370 and the peripheral screws 380 may be essentially the same as the central screw 170 (FIG. 4) and the peripheral screws 180 (FIG. 4) described above. The central screw 370 may be aligned along the fixation axis FA. The at least one peripheral screw 380 may be inserted into and through the peripheral bores 343 (FIG. 38) in the baseplate 340 to engage a patient's bone, such as, the glenoid, to assist with securing the baseplate 340 to the patient's bone.

As will be appreciated with reference to FIGS. 28 and 29, the glenoid implant system 300 is operably configured in this illustrated embodiment to provide a means for movably and pivotally connecting and rotating the mobile glenosphere 310 relative to the baseplate 340 so that the mobile glenosphere 310 is movable in a nonaxial direction relative to the fixation axis FA of the glenoid implant system 300. For example, the mobile glenosphere 310 may be pivotally rotatable in a first direction relative to the fixation axis FA and the baseplate 340 along the curved double headed arrow X, and in a second direction relative to the fixation axis FA and the baseplate along the curved double headed arrow X, (e.g., from one side to the other side of the baseplate). In this illustrated embodiment, due to the curved recess slot 330 and the guide 350, the central axis of the glenosphere 310 is pivotable from the first position to the second non-coaxial position. For example, with reference to FIG. 33, the central axis may be rotatable over a range of about 75 degrees, e.g., 35 degrees to each side of the position of the glenosphere 310 being centered relative to the baseplate 340. In other embodiments, the glenosphere 310 may be movable over an angle of 10 degrees, 20 degrees, 30 degrees, 45 degrees, 60 degrees, 70 degrees, or 90 degrees, a range between 10 degrees and 30 degrees, a range between 20 degrees and 50 degrees, a range between 30 degrees and 60 degrees, a range between 60 degrees and 80 degrees, or a range between 50 degrees and 90 degrees.

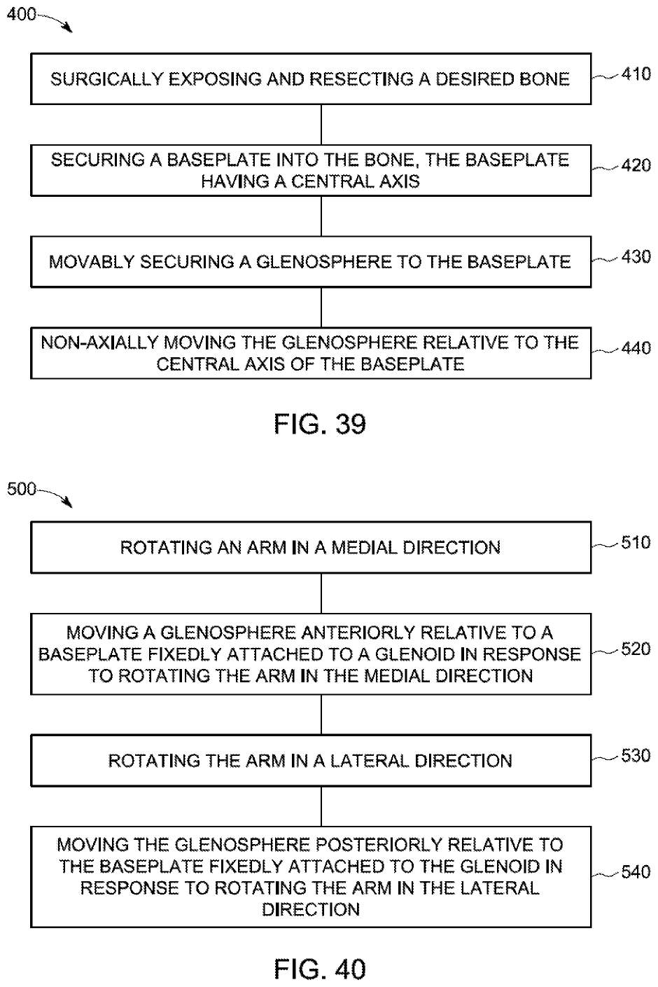

FIG. 39 illustrates a surgical method 400, according to an embodiment of the present disclosure. In this illustrated embodiment, the surgical method 400 may include, for example, at 410 surgically exposing and resecting a desired bone, at 420 securing a baseplate into the bone, the baseplate having a central axis, at 430 movably securing a glenosphere to the baseplate, and at 440, non-axially moving the glenosphere relative to the central axis of the baseplate.

FIG. 40 illustrates a method 500 for actuating a replacement joint, according to an embodiment of the present disclosure. In this illustrated embodiment, the method 500 may include, for example, at 510 rotating an arm of a patient in a medial direction, at 520 moving a glenosphere anteriorly relative to a baseplate fixedly attached to a glenoid in the patient in response to rotating the arm in the medial direction, at 530 rotating the arm in a lateral direction, and at 540 moving the glenosphere posteriorly relative to the baseplate fixedly attached to the glenoid in response to rotating the arm in the lateral direction.

In the various embodiments, the reverse prosthesis implant systems of the present disclosure having a movable glenosphere may be designed to fit the various patient anatomies that may be encountered. For example, a plurality of glenoid implant assemblies and humeral implant assemblies may be designed in multiple radii of curvature or having different concave surfaces, and depth options to allow for selecting the best fit for a given patient anatomy and corresponding defect.

From the present description, it will be appreciated that the technique of the present disclosure overcomes the unnatural motion introduced with conventional normal reverse total shoulder configurations can lead to limited range of motion and over-stretching of the muscles by allowing the glenoid component to move in a manner that allows the humerus to function similar to its natural range of motion. For example, the technique of the present disclosure allows the humerus to “roll” in place similar to anatomic kinematics. In addition, this may eliminate the risk of decreased range of motion and over-stretching of the soft tissue, while maintaining reverse shoulder kinematics and stability.

As may be recognized by those of ordinary skill in the art based on the teachings herein, numerous changes and modifications may be made to the above-described and other embodiments of the present disclosure without departing from the scope of the disclosure. The implants, screws, and other components of the devices and/or apparatus as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the devices and apparatus may include more or fewer components or features than the embodiments as described and illustrated herein. Accordingly, this detailed description of the currently-preferred embodiments is to be taken as illustrative, as opposed to limiting the disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The disclosure has been described with reference to the preferred embodiments. It will be understood that the architectural and operational embodiments described herein are exemplary of a plurality of possible arrangements to provide the same general features, characteristics, and general apparatus operation. Modifications and alterations will occur to others upon a reading and understanding of the preceding detailed description. It is intended that the disclosure be construed as including all such modifications and alterations.

I claim:

1. A glenoid implant system for use in a reverse total shoulder replacement in a patient, said glenoid implant system comprising:
a glenosphere comprising a body having a first portion and a second portion, said first portion having an articular surface, and said body defining a central axis extending from said first portion to said second portion;
a baseplate fixedly attachable to a glenoid of the patient, said baseplate having a body defining a central axis;
a connector for movably connecting said glenosphere to said baseplate; and
wherein said glenosphere and said baseplate are operably configured so that said central axis of said glenosphere is movable non-axially relative to said central axis of said baseplate.

2. The glenoid implant system of claim 1, wherein said central axis of said glenosphere is tiltably movable relative to said central axis of said baseplate.

3. The glenoid implant system of claim 1, wherein said central axis of said glenosphere is tiltably movable over a range between 10 degrees and 30 degrees relative to said central axis of said baseplate.

4. The glenoid implant system of claim 1, wherein said second portion of said glenosphere comprises a convex surface facing said baseplate.

5. The glenoid implant system of claim 1, wherein said body of said glenosphere comprises a passageway from said first portion to said second portion, and said connector is extendable through said passageway for movably connecting said glenosphere relative to said baseplate.

6. The glenoid implant system of claim 5, wherein said passageway comprises a cylindrical portion, a track portion, and expanded portion extending from said cylindrical portion to said track portion.

7. The glenoid implant system of claim 6, wherein said expanded portion of said passageway comprises parallel sidewalls.

8. The glenoid implant system of claim 1, wherein said central axis of said glenosphere is movable around a pivot axis.

9. The glenoid implant system of claim 8, wherein said body of said glenosphere comprises a passageway from said first portion to said second portion, and said connector is extendable through said passageway for pivotally connecting said glenosphere about said pivot axis.

10. The glenoid implant system of claim 8, wherein said second portion of said glenosphere comprises a flat surface facing said baseplate.

11. The glenoid implant system of claim 8, wherein said second portion of said glenosphere comprises a curved slot facing said baseplate, and said baseplate comprises a post receivable in said curved slot.

12. The glenoid implant system of claim 8, wherein said central axis of said glenosphere is movable around said pivot axis between 60 degrees and 80 degrees.

13. The glenoid implant system of claim 1, further comprising a humeral implant system.

14. The glenoid implant system of claim 13, wherein said humeral implant system comprises a humeral cup and a humeral stem.

15. The glenoid implant system of claim 14, wherein said glenoid implant system comprises a central screw and a plurality of peripheral screws for securing said baseplate to the glenoid of the patient.

16. A glenoid implant system for use in a reverse total shoulder replacement of a patient, said glenoid implant system comprising:
a glenosphere comprising an articular surface;
a baseplate fixedly attachable to a glenoid of the patient, said baseplate having a central axis; and
a means for movably connecting said glenosphere to said baseplate so that said glenosphere is movable non-coaxially relative to said baseplate.

17. The glenoid implant system of claim 16, wherein said means for movably connecting is operable so that a central axis of said glenosphere is tiltable relative to said central axis of said baseplate.

18. The glenoid implant system of claim 16, wherein said means for movably connecting is operable so that said central axis of said glenosphere is tiltable along a plane relative to said baseplate.

19. The glenoid implant system of claim 16, wherein a central axis of said glenosphere is tiltable relative to said central axis of said baseplate over a range between 30 degrees and 60 degrees.

20. The glenoid implant system of claim 16, wherein said glenosphere is rotatable about a pivot axis offset from said central axis of said baseplate.

21. The glenoid implant system of claim 20, wherein said central axis of said glenosphere is rotatable along a curve relative to said baseplate.

22. The glenoid implant system of claim 20, wherein a central axis of said glenosphere is rotatable about a pivot axis over a range between 50 degrees and 90 degrees.

23. The glenoid implant system of claim 16, further comprising a humeral implant system.

24. The glenoid implant system of claim 23, wherein said humeral implant system comprises a humeral cup and a humeral stem.

25. The glenoid implant system of claim 24, wherein said glenoid implant system comprises a central screw and a plurality of peripheral screws for securing said baseplate to the glenoid of the patient.

26. A method comprising:
surgically exposing and resecting a desired bone;
securing a baseplate to the bone, the baseplate having a central axis;
movably securing a glenosphere to the baseplate; and
non-axially moving a central axis of the glenosphere relative to the central axis of the baseplate.

27. The method of claim 26, wherein the non-axially moving comprises tilting the central axis of the glenosphere relative to the baseplate.

28. The method of claim 26, wherein the non-axially moving comprises tilting the central axis of the glenosphere along a plane relative to the baseplate.

29. The method of claim 26, wherein the non-axially moving comprises rotating the central axis of the glenosphere about a pivot axis offset from the central axis of the baseplate.

30. The method of claim 26, wherein the non-axially moving comprises rotating the central axis of the glenosphere along a curve relative to the baseplate.

31. A method comprising:
rotating an arm of a patient in a medial direction;
moving a glenosphere anteriorly relative to a baseplate that is fixedly attached to a glenoid of the patient in response to moving the arm in the medial direction;
rotating the arm in a lateral direction; and
moving the glenosphere posteriorly relative to the baseplate that is fixedly attached to the glenoid in response to moving the arm in the lateral direction.

32. The method of claim 31, wherein the central axis of the glenosphere is tiltable relative to the baseplate.

33. The method of claim 31, wherein the central axis of the glenosphere is tiltable along a plane relative to the baseplate.

34. The method of claim 31, wherein the glenosphere is rotatable about a pivot axis offset from a central axis of the baseplate.

35. The method of claim 31, wherein the central axis of the glenosphere is rotatable along a curve relative to the baseplate.

36. The glenoid implant system of claim 1, wherein said central axis of said glenosphere is angularly movable relative to said central axis of said baseplate.

37. A glenoid implant system for use in a reverse total shoulder replacement of a patient, said glenoid implant system comprising:
a glenosphere comprising an articular surface;
a baseplate fixedly attachable to a glenoid of the patient, said baseplate having a central axis; and
a means for movably connecting said glenosphere to said baseplate so that said glenosphere is movable non-coaxially relative to said baseplate.

38. The glenoid implant system of claim 37, wherein said means for movably connecting is operable so that said central axis of said glenosphere is tiltable relative to said baseplate.

39. The glenoid implant system of claim 37, wherein said means for movably connecting is operable so that said central axis of said glenosphere is tiltable along a plane relative to said baseplate.

40. The glenoid implant system of claim 37, wherein said central axis of said glenosphere is tiltable over an angle of 10 degrees, 20 degrees, 30 degrees, 45 degrees, 60 degrees, 70 degrees, or 90 degrees, a range between 10 degrees and 30 degrees, a range between 20 degrees and 50 degrees, or a range between 30 degrees and 60 degrees.

41. The glenoid implant system of claim 37 wherein said glenosphere is rotatable about a pivot axis offset from said central axis of said baseplate.

42. The glenoid implant system of claim 37, wherein said central axis of said glenosphere is rotatable along a curve relative to said baseplate.

43. The glenoid implant system of claim 37, wherein said central axis of said glenosphere is rotatable about a pivot axis over an angle of 10 degrees, 20 degrees, 30 degrees, 45 degrees, 60 degrees, 70 degrees, or 90 degrees, a range between 10 degrees and 30 degrees, a range between 20 degrees and 50 degrees, or a range between 30 degrees and 60 degrees.

44. The glenoid implant system of claim 37, further comprising a humeral implant system.

45. The glenoid implant system of claim 1, further comprising a humeral implant system comprising a humeral cup and a humeral stem.

46. The glenoid implant system of claim 1, wherein said glenoid implant system comprises a central screw and a plurality of peripheral screws for securing said baseplate to the glenoid.
